(19) United States
(12) Patent Application Publication
(10) Pub. No.: US 2008/0076977 A1
(43) Pub. Date: Mar. 27, 2008

(54) Patient monitoring device snapshot feature system and method

(75) Inventors: Paul D. Mannheimer, Danville, CA (US); Darius Eghbal, Oakland, CA (US)

Correspondence Address: Covidien;IP Counsel - Respiratory & Monitoring Solutions, 60 Middletown Avenue, North Haven, CT, 06473, US

(73) Assignee: Nellcor Puritan Bennett Inc.

(21) Appl. No.: 11/527,057

(22) Filed: Sep. 26, 2006

(51) Int. Cl. A61B 5/00

(52) U.S. Cl. 600/301

(57) ABSTRACT

Embodiments of the present invention relate to a monitoring system. The monitoring system may comprise a monitor configured to receive input relating to patient physiological parameters, a screen configured to display data corresponding to the patient physiological parameters, and a snapshot display feature configured to display a snapshot view on the screen, wherein the snapshot view includes fixed or stabilized graphical features that correspond to measurements of the patient physiological parameters.

### BACKGROUND OF THE INVENTION

1. Field of the Invention

The present invention relates generally to a user-interface feature for a patient monitoring device. In particular, the present invention relates to a display feature that facilitates data observation with a patient physiological data monitoring instrument.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Patient monitors include medical devices that facilitate measurement and observation of patient physiological data. For example, pulse oximeters are a type of patient monitor. A typical patient monitor cooperates with a sensor to continually detect and display a patient's vital signs (e.g., temperature, pulse rate, respiratory rate) and/or other physiological measurements (e.g., water content of tissue, blood oxygen level) for observation by a user (e.g., clinician). For example, pulse oximeters are generally utilized with related sensors to detect and monitor a patient's functional arterial blood oxygen saturation (i.e., SpO₂) and pulse rate. Other types of patient monitors may be utilized to detect and monitor other physiological parameters. The use of patient monitors may improve patient care by facilitating continuous supervision of a patient without continuous attendance by a human observer (e.g., a nurse or physician).

A patient monitor may include a screen that displays information relating to operation and use of the patient monitor. A typical patient monitor screen may display operational data that is instructive and that facilitates operation of the monitor by a user. For example, the operational data may include status indicators and instructional data relating to the monitor itself and/or monitor applications (e.g., a power indicator, an alarm silenced icon, and a battery low indicator). The screen may also display measurement data from a patient being monitored. For example, the measurement data may include information relating to a physiological feature of the patient being monitored (e.g., a pulse amplitude indicator, a pulse rate trend, a plethysmographic waveform trend).

A user or caregiver may utilize a patient monitor to track a patient's progress or to simply observe the patient's status. For example, a user may observe certain values on the monitor screen and then transcribe the values onto the patient's chart for later reference. Additionally, a user may observe data on the monitor screen and make a status determination based on recent indicators. However, because of the quantity of information on a typical monitor screen and because the data on the screen is often displayed in real-time, it can be difficult and/or inconvenient for a user to select appropriate values for making a determination or for reporting (e.g., transcribing on patient's chart, saving to a central memory).

### BRIEF DESCRIPTION OF THE DRAWINGS

Advantages of the invention may become apparent upon reading the following detailed description and upon reference to the drawings in which:

FIG. 1 is a perspective view of a patient monitor in accordance with an exemplary embodiment of the present invention;

FIG. 2 is a perspective view of the patient monitor in a system with separate devices in accordance with an exemplary embodiment of the present invention;

FIG. 3 is a representation of a view that may be frozen in accordance with an exemplary embodiment of the present invention;

FIG. 4 is a representation of a view that may be frozen in accordance with an exemplary embodiment of the present invention; and

FIG. 5 is a representation of a blip-bar view with an activated snapshot window displayed therein in accordance with an exemplary embodiment of the present invention.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present invention are directed to a patient monitor (e.g., a pulse oximeter) with a user interface that allows a user to obtain a snapshot view or display of patient information. As used herein and described below, the term “snapshot view” refers to some or all information in a display (e.g., graphical display on a monitor) that is normally changing in real-time operation to be held fixed (unchanging) for a brief or extended period of time sufficient for a user to more readily interpret, comprehend, or otherwise understand its content. This snapshot view may be presented on a screen of the patient monitor or a screen in communication with the patient monitor. The snapshot view may assist a user in obtaining relevant data for tracking a patient's progress or in obtaining a representative spot reading of the patient's status. Specifically, the snapshot view may facilitate viewing of stable measurement values by displaying a frozen view of the standard screen data and/or by displaying a steady view of selected data values (e.g., an average heart rate for a previous time period). For example, the snapshot display information may provide an unchanging view of the most recent instantaneous values displayed in a normal view, an unchanging view of a value from a past time, and/or an average of values calculated from a monitoring epoch (e.g., the previous 15, 30, or 60 seconds). The displayed information could include textual data, numeric data, graphical data, or a combination of data types.

The snapshot view may be generated by a snapshot display feature of the monitor. The snapshot display feature may include hardware, software, or a combination of hardware and software that is configured to provide the snapshot view discussed above and described in further detail below. For example, in one embodiment, the snapshot display feature provides a frozen view of the normal view displayed by the monitor. It should be noted that the normal view may be provided by a normal operation display feature of the monitor. The normal operation display feature may include hardware, software, or a combination of both that is configured to display visual data (e.g., real-time measurements of physiological parameters) on a display screen. A user may activate the snapshot view by manipulating an activation mechanism (e.g., pressing a button or navigating a menu). For example, the user may utilize a menu to select specific features (e.g., a blip-bar corresponding to pulse amplitude) corresponding to specific parameters to freeze. Additionally, in some embodiments, the user may select a time frame for the data (e.g., one hour ago, the last 30 seconds).

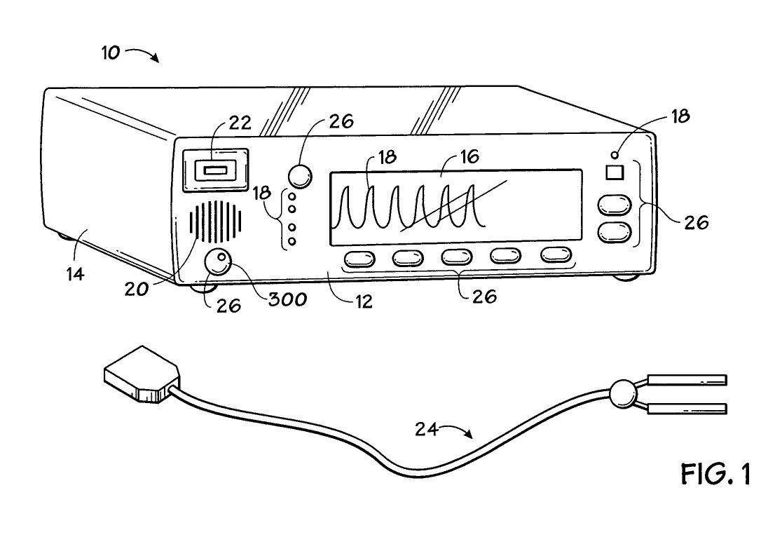

FIG. 1 is a perspective view of a patient monitor 10 in accordance with an exemplary embodiment of the present invention. Specifically, the patient monitor 10 illustrated by FIG. 1 is a pulse oximeter that is configured to detect and monitor blood oxygen saturation levels, pulse rate, and so forth. It should be noted that while the illustrated embodiment includes a pulse oximeter, other embodiments of the present invention may include different types of patient monitors 10. For example, the patient monitor 10 may be representative of a vital signs monitor, a critical care monitor, an obstetrical care monitor, or the like.

The illustrated patient monitor 10 includes a front panel 12 coupled to a body 14 of the monitor 10. The front panel 12 includes a display screen 16 and various indicators 18 (e.g., indicator lights and display screen graphics) that facilitate operation of the monitor 10 and observation of a patient's physiological metrics (e.g., pulse rate). Some of the indicators 18 are specifically provided to facilitate monitoring of a patient's physiological parameters. For example, the indicators 18 may include representations of the most recently measured values for SpO₂, pulse rate, and pulse amplitude. Other indicators 18 may be specifically provided to facilitate operation of the monitor 10. For example, the indicators 18 may include an A/C power indicator, a low battery indicator, an alarm silence indicator, a mode indicator, and so forth. The front panel 12 also includes a speaker 20 for emitting audible indications (e.g., alarms), a sensor port 22 for coupling with a sensor 24 (e.g., a temperature sensor, a pulse oximeter sensor) and other standard monitor features.

Additionally, the front panel 12 includes various activation mechanisms 26 (e.g., buttons and switches) to facilitate management and operation of the monitor 10. For example, the front panel 12 may include soft keys (e.g., keys with varying functions), a power switch, adjustment buttons, an alarm silence button, and so forth. It should be noted that in other embodiments, the indicators 18 and activation mechanisms 26 may be arranged on different parts of the monitor 10. In other words, the indicators 18 and activation mechanisms 26 are not necessarily located on the front panel 12 in accordance with present embodiments.

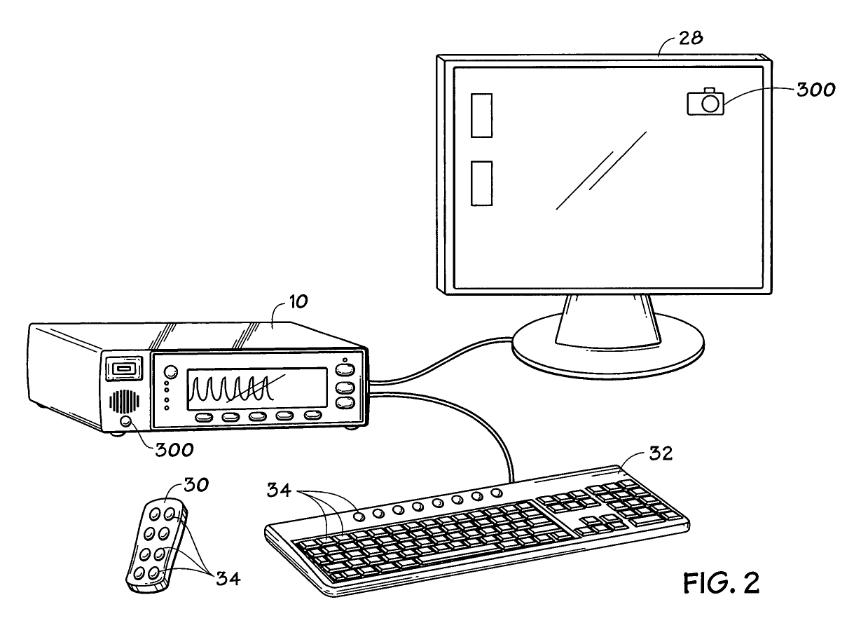

In some embodiments, as illustrated in FIG. 2, the monitor 10 cooperates with separate devices, such as a separate screen 28, a wireless remote 30, and/or a keyboard 32. These separate devices may include some of the indicators 18 and activation mechanisms 26 described above. For example, buttons 34 on the remote 30 and/or keyboard 32 may operate as activation mechanisms 26. Specifically, for example, the buttons 34 may cause the monitor 10 to perform specific operations (e.g., power up, adjust a setting, silence an alarm) when actuated on the separate device. Similarly, the indicators 18 may not be directly disposed on the monitor 10. For example, the indicators 18 may include icons, indicator lights, or graphics on the separate screen 28 (e.g., a computer screen). It should be noted that the separate screen 28 and/or the keyboard 32 may communicate directly or wirelessly with the monitor 10.

As briefly set forth above, embodiments of the present invention include a snapshot display feature that may allow a user to briefly freeze and/or open a display presented by the monitor 10 such that the display includes fixed indications of previous physiological measurements. This snapshot display feature may facilitate a user's observation of certain metrics (e.g., pulse rate, SpO₂) by eliminating or reducing the frequency of real-time updates and allowing the user to view steady or unchanging values. It should be noted that the snapshot view presented by the monitor 10 may be displayed by the monitor 10 on the display screen 16 or the separate screen 28.

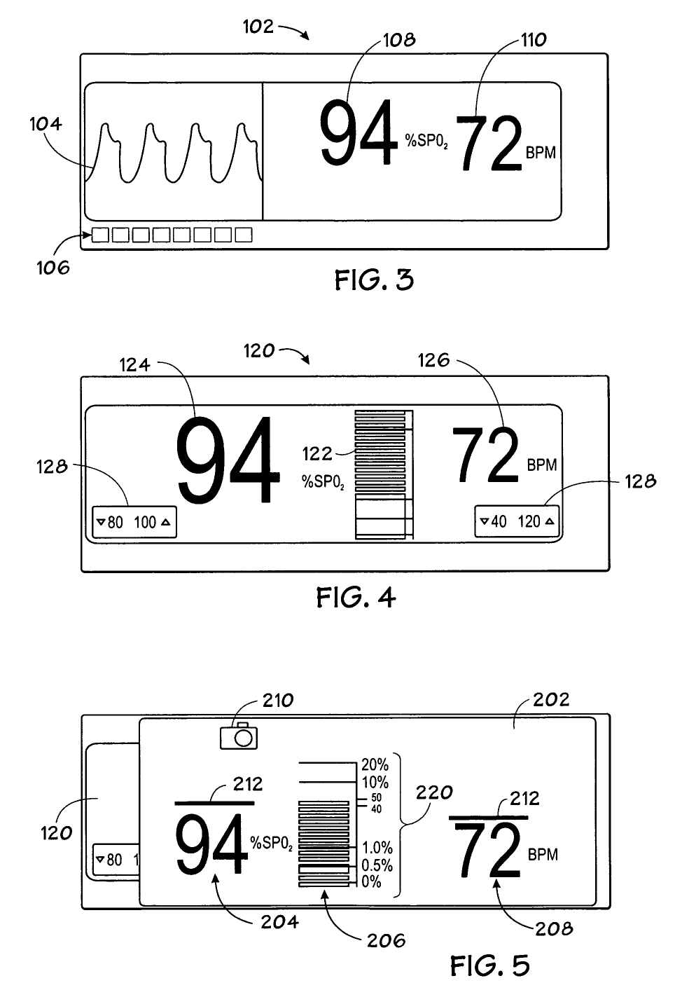

In one embodiment, the snapshot view feature simply freezes a real-time view in response to activation by a user (e.g., a button press). For example, FIGS. 3 and 4 include representations of exemplary views that may be frozen by the snapshot feature to facilitate observation. FIG. 3 includes a real-time waveform view 102 that displays a “wiper bar” plethysmographic waveform 104, a menu bar 106, a measured SpO₂ value 108, and a measured pulse rate 110. FIG. 4 includes a blip-bar view 120 that displays a pulse amplitude blip bar 122, a measured SpO₂ value 124, a measured pulse rate 126, and upper and lower SpO₂ and pulse rate limits 128. During normal operation, the graphic, textual, and numeric display features of the views 102 and 120 may be continually updated with the most recent measured values, and this continual updating can make it difficult for a user to observe a representative set of data. Accordingly, embodiments of the present invention enable the user to freeze or stabilize the views 102 and 120 by utilizing the snapshot display feature, thus enabling the observation of a stable or fixed set of data. In some embodiments, all or some of the display features (e.g., pulse amplitude blip bar 122, measured SpO₂ value 124, measured pulse rate 126) of a particular view (e.g., blip-bar view 120, real-time waveform view 102) may be frozen based on a user selection (e.g., pressing a button).

In some embodiments, the snapshot display feature does not merely freeze a normal operating view (e.g., a real-time view). For example, the snapshot display and the normal operating view may not include the same information and/or layout. Indeed, the snapshot view may include additional or substantially different information and graphic positioning. For example, the snapshot view may include additional scaling information, textual labels, and so forth. Further, the snapshot view may replace real-time data values with average data values and so forth. Such distinctions between normal and snapshot views may facilitate informed observations. Indeed, not only may the snapshot view directly provide additional information but, the differences between views may indicate the status of the observation to a user. For example, the difference between views may alert a viewer to the fact that he or she is not observing real-time values.

The snapshot display feature may launch a graphic or snapshot view within a real-time display or as a separate display, wherein the graphic includes a fixed or stabilized (e.g., slowly updated) data set to facilitate user observation. For example, FIG. 5 includes a representation of the blip-bar view 120 with an activated snapshot window 202 being displayed therein. The snapshot window 202 includes frozen or stabilized data to facilitate observation of a representative data set (e.g., a set of physiological metrics). As noted above, the snapshot display feature may provide additional information that is not shown during normal operation (e.g., real-time monitoring). For example, the snapshot window 202 in FIG. 5 includes a numeric display of recent average SpO₂ values 204, a graphical display of recent average pulse amplitude values 206, and a numeric display of recent average pulse rate values 208. These values for recent average data (e.g., SpO₂ 204, pulse amplitude 206, and pulse rate 208) may be calculated from a recent monitoring epoch (e.g., comparable to the previous 15, 30, or 60 seconds).

It should be noted that to further clarify that the snapshot view is not a normal view, the snapshot view may include a distinguishing visual feature (e.g., a textual indicator, an iconic display). For example, a camera icon 210 may be included in the snapshot window 202 of FIG. 5 to clearly indicate that that the type of view being depicted is a snapshot view. Further, a bar 212 may be placed over the numeric values in the snapshot window 202 to clarify that the displayed values 204 and 208 are averages. Use of a bar to indicate an average is common practice in math and science fields. Such an indication may also be accomplished with text (e.g., “30-sec average”). Similar indications may be used for different data types. Further, such indications may serve multiple purposes. Indeed, like the camera icon 210, these indications may alert a user to the nature of the of snapshot window 202. In other words, distinguishing features such as text, symbols, and icons may make the user aware that the monitor 10 is not in its normal mode of operation (e.g., real-time monitoring).

In some embodiments, the snapshot display feature provides additional data when activated to allow for a less cluttered display during normal operation (e.g., real-time monitoring). For example, a display generated by the snapshot feature may include scaling values 220 that are not shown in a real-time display (e.g., 102 and 120). Indeed, this is actually illustrated in FIGS. 4 and 5. The blip-bar view 120 in FIG. 4 does not include scaling values of pulse amplitude adjacent the blip bar 122. However, the snapshot window 202 in FIG. 5 includes a scale 220 with major and minor tick marks and corresponding values adjacent the graphical display of recent average pulse amplitude 206, which correlates to the blip bar 122. While the snapshot window 202 and the blip-bar view 120 are different depictions of pulse amplitude measurements, the scaling may be equivalent between the two depictions. Thus, a user may be educated as to the meaning and scaling of the graphical depiction of the measured variable (e.g., pulse amplitude) by activating the snapshot feature, without including permanent additional clutter on the real-time display.

Embodiments of the present invention include specific applications wherein the snapshot feature may be activated and/or deactivated to display and remove scaling information, labels, instructions, and so forth to reduce clutter and facilitate user comprehension. Graphical forms including graphic and textual indications of scaling may be added or removed from a display by activating or deactivating (e.g., selecting and icon, actuating a button) the snapshot display feature. For example, in some embodiments text indicators may be more meaningful than numeric values because related variables are not universally understood. In a specific example, it may be desirable to utilize text indicators of pulse amplitude (e.g., “very weak,” “weak,” “normal,” and “large”). However, including such text in the standard display may cause confusion by cluttering the presentation of measured data. Accordingly, present embodiments can reduce this clutter by allowing a user to essentially hide it from view during normal operation by deactivating the snapshot view. In another example, minor scaling and/or tick mark labels may be added adjacent a graphical form (e.g., the blip bar 122) by the snapshot feature when it is activated. This facilitates reading a value from the scale. Further, the minor scaling and/or tick mark labels may be removed by deactivating the snapshot feature to facilitate uncluttered real-time observation. It should be noted that, in some embodiments, the information displayed by activating the snapshot feature may overlay the real-time data.

As illustrated in FIG. 1 and FIG. 2, embodiments of the present invention may include a snapshot view activation feature 300 (e.g., button, icon) that can be selected or actuated to activate a snapshot display (e.g., freeze frame, additional display features). The snapshot view activation feature 300 may be disposed directly on the monitor 10 or incorporated with related devices. For example, the snapshot view activation feature 300 may include an icon on the separate screen 28, a button on the wireless remote 30, or a button on the keyboard 32. The snapshot view activation feature 300 may include a distinctive visual indicator that informs a user of its purpose. For example, a representation such as the camera icon 210 may be incorporated by the snapshot view activation feature 300 as a graphic on a button or as an icon. Indeed, in one embodiment the snapshot view activation feature 300 may include the camera icon 210 as a selectable graphic in a display provided by the monitor 10. In some embodiments, the snapshot view activation feature 300 may freeze the entire view when selected or it may freeze certain features in the view. For example, the snapshot view activation feature 300 may simply freeze a blip-bar that corresponds to a particular physiological parameter without freezing other features of the view. In this case, the snapshot view activation feature 300 may include both an icon that indicates the snapshot function (e.g., icon 210) and an icon indicating what feature will be frozen.

In accordance with present embodiments, upon activation of the snapshot display feature (e.g., via actuation of the snapshot view activation feature 300), the monitor 10 displays a snapshot display view for a designated amount of time (e.g., 10 seconds) before the standard display (e.g., real-time monitoring) automatically returns to the screen. For example, by activating the display feature, the monitor 10 may temporarily modify the display to show numeric, textual, or graphic features that facilitate data observation, and then automatically revert to a normal mode after a brief time-out period. This may facilitate reduction of clutter in the normal mode of operation and ensure that the view is returned to normal operation. In other embodiments a user may be required to toggle between displaying the snapshot view and the standard view. For example, if the snapshot display feature is activated by depressing a button, deactivation may be achieved by pressing the button again. Some embodiments may incorporate both delayed automatic reversion and toggle features.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A monitoring system, comprising:
a monitor configured to receive input relating to patient physiological parameters;
a screen configured to display data corresponding to the patient physiological parameters; and
a snapshot display feature configured to display a snapshot view on the screen, wherein the snapshot view includes fixed or stabilized graphical features that correspond to measurements of the patient physiological parameters.

2. The system of claim 1, comprising a normal operation display feature configured to provide a real-time view of measured values of the patient physiological parameters.

3. The system of claim 2, wherein the snapshot view includes fixed graphical representations of the most recent measured values provided in the real-time view.

4. The system of claim 2, wherein the snapshot view includes fixed graphical representations of measured values provided in the real-time view for a time selected by a user.

5. The system of claim 2, wherein the snapshot view includes stabilized graphical representations of the measured values provided in the real-time view.

6. The system of claim 1, wherein the snapshot view includes an icon indicative of activation of the snapshot display feature.

7. The system of claim 1, wherein the snapshot view includes a graphical representation of an average of measured values of the patient physiological parameters.

8. The system of claim 7, wherein the measured values were measured within a time period selected by a user.

9. The system of claim 1, comprising a symbol indicative of a nature of the graphical features.

10. The system of claim 9, wherein the symbol indicates that the measurements of the patient physiological parameters include an average value of the measurements.

11. The system of claim 1, wherein the snapshot view includes a snapshot window.

12. The system of claim 1, wherein the snapshot view includes graphical features representative of scaling values, labels, instructions, or a combination of scaling values, labels, and instructions.

13. The system of claim 1, wherein the fixed or stabilized graphical features include representations of numerical data.

14. The system of claim 13, wherein the fixed or stabilized graphical features include Arabic numerals.

15. A method, comprising:
receiving input relating to patient physiological parameters;
displaying visual data corresponding to the patient physiological parameters in a normal view; and
displaying a snapshot view including fixed or stabilized graphical features that correspond to measurements of the patient physiological parameters when a snapshot display feature is activated.

16. The method of claim 15, comprising displaying real-time measured values of the patient physiological parameters in the normal view.

17. The method of claim 16, wherein the snapshot view includes fixed graphical representations of the most recent real-time, measured values.

18. The method of claim 16, wherein the snapshot view includes fixed graphical representations of measured values provided in the real-time view for a time selected by a user.

19. The method of claim 16, wherein the snapshot view includes stabilized graphical representations of the real-time, measured values of the real-time view.

20. The method of claim 15, comprising displaying an icon indicative of the nature of the snapshot view in the snapshot view.

21. The method of claim 15, comprising displaying a graphical representation of an average of measured values in the snapshot view.

22. The method of claim 15, comprising displaying a snapshot window as the snapshot view while displaying the normal view in the background.

23. The method of claim 15, comprising displaying graphical features representative of scaling values, labels, instructions, or a combination of scaling values, labels, and instructions as components of the snapshot view.

24. The method of claim 15, comprising displaying the snapshot view for a designated amount of time and automatically closing the snapshot view.

25. The method of claim 15, comprising displaying the snapshot view when a toggle feature is activated and closing the snapshot view with the toggle feature is deactivated.

26. The method of claim 15, comprising displaying Arabic numerals as the fixed or stabilized graphical features that correspond to the measurements of the patient physiological parameters.

27. A method, comprising:
displaying a real-time measurement of a physiological parameter on a monitor screen in a normal view; and
displaying fixed or stabilized value of the real-time measurement of the physiological parameter on the monitor screen when a snapshot display feature is activated.

28. The method of claim 27, comprising hiding the fixed or stabilized value when the snapshot display feature is deactivated.

29. The method of claim 27, wherein the snapshot display feature is automatically deactivated after an amount of time.

30. The method of claim 27, wherein the fixed or stabilized value includes a graphical feature representative of scaling values, labels, or instructions.

31. A method, comprising:
causing a monitor to display a snapshot view including fixed or stabilized graphical features that correspond to measurements of patient physiological parameters by activating a snapshot display feature.

32. The method of claim 31, comprising selecting an icon that activates the snapshot display feature.

33. The method of claim 31, comprising depressing a button that activates the snapshot display feature.
